(19) United States
(12) Patent Application Publication
(10) Pub. No.: US 2018/0282794 A1
(43) Pub. Date: Oct. 4, 2018

(54) Sample Preparation Vessels, Microfluidic Circuits, and Systems and Methods for Sample Preparation, Extraction, and Analysis

(71) Applicant: ENVIROLOGIX INC., PORTLAND, ME (US)

(72) Inventor: DANIEL SHAFFER, PORTLAND, ME (US)

(73) Assignee: ENVIROLOGIX INC., PORTLAND, ME (US)

(21) Appl. No.: 15/521,869

(22) PCT Filed: Oct. 21, 2015
(86) PCT No.: PCT/US15/56641

§ 371 Date: Apr. 25, 2017

Related U.S. Application Data
(60) Provisional application No. 62/069,800, filed on Oct. 28, 2014.

(51) Int. Cl. C12Q 1/6837; C12Q 1/686; C12Q 1/6865; C12Q 1/6867

(52) U.S. Cl. CPC C12Q 1/6837; C12Q 1/686; C12Q 2563/107; C12Q 1/6867; C12Q 1/6865

(57) ABSTRACT

The invention generally provides a sample preparation vessel including a flexible substrate defining at least one sealable opening adapted and configured to receive a solid sample; at least one fitting; and at least one filter adjacent to the at least one fitting, the filter adapted and configured to permit extracted fluids to exit the vessel while retaining solid particles, as well as vessels, circuits, systems, and related methods for sample preparation, extraction, and analysis.

### CROSS REFERENCE TO RELATED APPLICATIONS

This application claims the benefit of and priority to U.S. Provisional Application Ser. No. 62/069,800, filed Oct. 28, 2014, the contents of which are incorporated herein by reference in their entirety.

### BACKGROUND OF THE INVENTION

As biotechnology evolves, many processes that were once performed in controlled laboratory environments are now sought in the field. This poses challenges not only in ensuring robustness in the face of variable environmental conditions and preventing cross-contamination, but also in ensuring reliable operation by users that may not be scientifically trained.

### SUMMARY OF THE INVENTION

One aspect of the invention provides a sample preparation vessel including: a flexible substrate defining at least one sealable opening adapted and configured to receive a solid sample; at least one fitting; and at least one filter adjacent to the at least one fitting, the filter adapted and configured to permit extracted fluids to exit the vessel while retaining solid particles.

Another aspect of the invention provides a microfluidic circuit including: a fluidic path;

a first row of windows, each window including a chamber and an optical lens dome on a first surface of the microfluidic circuit; and an outlet adapted and configured for coupling with additional rows of windows.

Another aspect of the invention provides a system including: a first port in fluid communication with at least one fluid reservoir and adapted and configured for removable coupling with a sample preparation vessel, the one or more ports collectively; a second port adapted and configured to receive a sample from a sample mixing circuit; a first receptacle adapted and configured to receive the sample preparation vessel; and a second receptacle adjacent to the first receptacle. The second receptacle is adapted and configured to receive the sample mixing circuit and hold the sample mixing circuit in fluid communication with the sample preparation vessel.

This aspect of the invention can have a variety of embodiments. The system can further include a homogenizer adapted and configured to press against the sample preparation vessel and substantially homogenize the contents thereof. The homogenizer can include a rack and pinion gear.

The system can include an array of optical imaging devices, each adapted to image at least one row of windows of the sample mixing circuit. The system can include a third receptacle including an interface for an additional array of optical imaging devices.

The system can include a compression device adapted and configured to compress one or more blisters on the sample mixing circuit to release one or more reagents. The compression device can be a roller.

Another aspect of the invention provides a method for extracting an analyte from a sample. The method includes: introducing the sample into a sample preparation vessel as described herein; and mixing the sample with a buffer capable of extracting and/or solubilizing the analyte in the sample preparation vessel, thereby extracting an analyte from a sample.

Another aspect of the invention provides a method for extracting an analyte from a solid sample. The method includes: introducing the solid sample into a sample preparation vessel as described herein; mixing the sample with a buffer capable of solubilizing the analyte in the sample preparation vessel; and macerating or homogenizing the solid sample, thereby extracting an analyte from the solid sample.

This aspect of the invention can have a variety of embodiments. The sample or solid sample can be a biological sample or an environmental sample. The sample or solid sample can be a seed, plant tissue, or plant part. The analyte can be a DNA, RNA, nucleic acid, protein, carbohydrate, and/or lipid.

Another aspect of the invention provides a method of detecting an analyte. The method includes: introducing a sample comprising the analyte into the mixing chamber of the microfluidic circuit as described herein, wherein the mixing chamber comprises one or more reagents for the reaction; and detecting the analyte in a window of the microfluidic circuit.

Another aspect of the invention provides a method of detecting a target nucleic acid molecule. The method includes: introducing a sample comprising a target nucleic acid molecule into the mixing chamber of the microfluidic circuit as described herein, wherein the mixing chamber comprises one or more reagents for amplifying the target nucleic acid; and detecting the target nucleic acid molecule in a window of the microfluidic circuit.

This aspect of the invention can have a variety of embodiments. The microfluidic circuit can include one or more blisters in fluid connection with the mixing chamber. Compression of one or more blisters can introduce one or more reagents into the mixing chamber.

The reagents can include one or more of a nickase, DNA polymerase, RNA polymerase, dNTPs, primer, probe, enzyme, and/or reaction buffer. The target nucleic acid can be DNA or RNA. The reaction can PCR, qPCR, an isothermal nucleic acid amplification reaction, Nicking and Extension Amplification Reaction (NEAR), Rolling Circle Amplification (RCA), Helicase-Dependent Amplification (HDA), Loop-Mediated Amplification (LAMP), Strand Displacement Amplification (SDA), Transcription-Mediated Amplification (TMA), Self-Sustained Sequence Replication (3SR), Nucleic Acid Sequence Based Amplification (NASBA), Single Primer Isothermal Amplification (SPIA), Q-Replicase System, or Recombinase Polymerase Amplification (RPA).

Another aspect of the invention provides a method of detecting an analyte in a sample. The method includes: extracting an analyte from a sample in the sample preparation vessel as described herein; mixing the analyte and one or more reagents in the sample mixing circuit of the system; and detecting the analyte using an optical imaging device of the system.

Another aspect of the invention provides a method of detecting one or more analytes in a sample. The method includes: extracting the one or more analytes from the sample in the sample preparation vessel as described herein; mixing the analytes and one or more preparation reagents in the sample mixing circuit of the system; introducing the mixture of analytes and preparation reagents into an array of chambers or windows comprising one or more detection reagents; and detecting the analytes using the array of optical imaging devices of the system.

Another aspect of the invention provides a method of detecting a target nucleic acid molecule in a sample. The method includes: extracting the target nucleic acid molecule from a sample in the sample preparation vessel as described herein; mixing the target nucleic acid molecule and one or more reagents in the sample mixing circuit of the system; amplifying the target nucleic acid molecule; and detecting the analyte using an optical imaging device of the system.

Another aspect of the invention provides a method of detecting one or more target nucleic acid molecules in a sample. The method includes: extracting the one or more target nucleic acid molecules from the sample in the sample preparation vessel as described herein; mixing the one or more target nucleic acid molecules and one or more preparation reagents in the sample mixing circuit of the system; introducing the mixture of target nucleic acid molecules and preparation reagents into an array of chambers or windows comprising one or more amplification and/or detection reagents; amplifying the target nucleic acid molecules in the array of chambers or windows; and detecting the analytes using the array of optical imaging devices of the system.

This aspect of the invention can have a variety of embodiments. The reagents can comprise one or more of a nickase, DNA polymerase, RNA polymerase, dNTPs, primer, probe, enzyme, and/or reaction buffer. The target nucleic acid can be DNA or RNA. The amplifying step can be by PCR, qPCR, an isothermal nucleic acid amplification reaction, Nicking and Extension Amplification Reaction (NEAR), Rolling Circle Amplification (RCA), Helicase-Dependent Amplification (HDA), Loop-Mediated Amplification (LAMP), Strand Displacement Amplification (SDA), Transcription-Mediated Amplification (TMA), Self-Sustained Sequence Replication (3SR), Nucleic Acid Sequence Based Amplification (NASBA), Single Primer Isothermal Amplification (SPIA), Q-Replicase System, or Recombinase Polymerase Amplification (RPA).

Each chamber or window can comprise a set of nucleic acid primers for amplifying the target nucleic acid. Each chamber or window can include a fluorescently labeled nucleic acid probe for detecting the target nucleic acid.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIGS. 1A-1C depict sample preparation, extraction, and analysis systems according to embodiments of the invention.

FIGS. 2A-2C depict sample preparation vessels according to embodiments of the invention.

FIG. 3 depicts a sample mixing circuit according to an embodiment of the invention.

FIGS. 4A-4C depict assay modules according to embodiments of the invention.

FIG. 5 depicts a detection module according to an embodiment of the invention.

FIGS. 6A and 6B depicts a rack-and-pinion homogenizer according to an embodiment of the invention.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

By “amplicon” is meant a polynucleotide generated during the amplification of a polynucleotide of interest. In one example, an amplicon is generated during a polymerase chain reaction.

The term “analyte” is meant any compound under investigation using an analytical method. In particular embodiments, analytes include any nucleic acid molecule, polypeptide, carbohydrate, lipid, small molecule, marker, or fragments thereof.

By “base substitution” is meant a substituent of a nucleobase polymer that does not cause significant disruption of the hybridization between complementary nucleotide strands.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “complementary” or “complementarity” is meant that a nucleic acid can form hydrogen bond(s) with another nucleic acid sequence by either traditional Watson-Crick or Hoogsteen base pairing. Complementary base pairing includes not only G-C and A-T base pairing, but also includes base pairing involving universal bases, such as inosine. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). To determine that a percent complementarity is of at least a certain percentage, the percentage of contiguous residues in a nucleic acid molecule that can form hydrogen bonds (e.g., Watson-Crick base pairing) with a second nucleic acid sequence is calculated and rounded to the nearest whole number (e.g., 12, 13, 14, 15, 16, or 17 nucleotides out of a total of 23 nucleotides in the first oligonucleotide being based paired to a second nucleic acid sequence having 23 nucleotides represents 52%, 57%, 61%, 65%, 70%, and 74%, respectively; and has at least 50%, 50%, 60%, 60%, 70%, and 70% complementarity, respectively). As used herein, “substantially complementary” refers to complementarity between the strands such that they are capable of hybridizing under biological conditions. Substantially complementary sequences have 60%, 70%, 80%, 90%, 95%, or even 100% complementarity. Additionally, techniques to determine if two strands are capable of hybridizing under biological conditions by examining their nucleotide sequences are well known in the art.

As used herein, “duplex” refers to a double helical structure formed by the interaction of two single stranded nucleic acids. A duplex is typically formed by the pairwise hydrogen bonding of bases, i.e., “base pairing”, between two single stranded nucleic acids which are oriented antiparallel with respect to each other. Base pairing in duplexes generally occurs by Watson-Crick base pairing, e.g., guanine (G) forms a base pair with cytosine (C) in DNA and RNA, adenine (A) forms a base pair with thymine (T) in DNA, and adenine (A) forms a base pair with uracil (U) in RNA. Conditions under which base pairs can form include physiological or biologically relevant conditions (e.g., intracellular: pH 7.2, 140 mM potassium ion; extracellular pH 7.4, 145 mM sodium ion). Furthermore, duplexes are stabilized by stacking interactions between adjacent nucleotides. As used herein, a duplex may be established or maintained by base pairing or by stacking interactions. A duplex is formed by two complementary nucleic acid strands, which may be substantially complementary or fully complementary. Single-stranded nucleic acids that base pair over a number of bases are said to “hybridize.”

“Detect” refers to identifying the presence, absence or amount of the analyte to be detected. In one embodiment, the analyte is a polynucleotide.

By “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “fragment” is meant a portion of a nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 10, 15, 20, 30, 40, 50, 60, 70, 80, 90, or 100 nucleotides. In one embodiment, the fragment comprises at least about 50, 75, 80, 85, 89, 90, or 100 nucleotides of a polynucleotide.

By “hybridize” is meant to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). Hybridization occurs by hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “isolated polynucleotide” is meant a nucleic acid (e.g., a DNA, RNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “melting temperature (Tm)” is meant the temperature of a system in equilibrium where 50% of the molecular population is in one state and 50% of the population is in another state. With regard to the nucleic acids of the invention, Tm is the temperature at which 50% of the population is single-stranded and 50% is double-stranded (e.g., intramolecularly or intermolecularly).

By “monitoring a reaction” is meant detecting the progress of a reaction. In one embodiment, monitoring reaction progression involves detecting polymerase extension and/or detecting the completion of an amplification reaction.

As used herein, “obtaining” as in “obtaining an agent” includes synthesizing, purchasing, or otherwise acquiring the agent.

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, 2′ modified nucleotides (e.g., 2′-O-methyl ribonucleotides, 2′-F nucleotides).

As used herein, “modified nucleotide” refers to a nucleotide that has one or more modifications to the nucleoside, the nucleobase, pentose ring, or phosphate group. For example, modified nucleotides exclude ribonucleotides containing adenosine monophosphate, guanosine monophosphate, uridine monophosphate, and cytidine monophosphate and deoxyribonucleotides containing deoxyadenosine monophosphate, deoxyguanosine monophosphate, deoxythymidine monophosphate, and deoxycytidine monophosphate. Modifications include those naturally occurring that result from modification by enzymes that modify nucleotides, such as methyltransferases. Modified nucleotides also include synthetic or non-naturally occurring nucleotides. Synthetic or non-naturally occurring modifications in nucleotides include those with 2′ modifications, e.g., 2′-O-methyl, 2′-methoxyethoxy, 2′-fluoro, 2′-hydroxyl (RNA), 2′-allyl, 2′-O-[2-(methylamino)-2-oxoethyl], 4′-thio, 4′-CH₂—O-2′-bridge, 4′-(CH₂)₂—O-2′-bridge, and 2′-O-(N-methylcarbamate) or those comprising base analogs.

By “nucleotide adduct” is meant a moiety that is bound covalently or otherwise fixed to a standard nucleotide base.

By “nicking agent” is meant a chemical entity capable of recognizing and binding to a specific structure in double stranded nucleic acid molecules and breaking a phosphodiester bond between adjoining nucleotides on a single strand upon binding to its recognized specific structure, thereby creating a free 3′-hydroxyl group on the terminal nucleotide preceding the nick site. In preferred embodiments, the 3′ end can be extended by an exonuclease deficient polymerase. Exemplary nicking agents include nicking enzymes, RNAzymes, DNAzymes, and transition metal chelators.

By “polymerase-arresting molecule” is meant a moiety associated with a polynucleotide template/primer that prevents or significantly reduces the progression of a polymerase on the polynucleotide template. Preferably, the moiety is incorporated into the polynucleotide. In one preferred embodiment, the moiety prevents the polymerase from progressing on the template.

By “polymerase extension” is meant the forward progression of a polymerase that matches incoming monomers to their binding partners on a template polynucleotide. As used herein, “primer-dimer” is meant a dimer of two monomer oligonucleotide primers. In the oligonucleotide primers of the invention, the 5′ tail regions of monomer primers dimerize.

By “semi-quantitative” is meant providing an estimate of relative quantity based on an internal control.

By “specific product” is meant a polynucleotide product resulting from the hybridization of primer oligonucleotides to a complementary target sequence and subsequent polymerase mediated extension of the target sequence.

By “substantially isothermal condition” is meant at a single temperature or within a narrow range of temperatures that does not vary significantly. In one embodiment, a reaction carried out under substantially isothermal conditions is carried out at a temperature that varies by only about 1-5° C. (e.g., varying by 1, 2, 3, 4, or 5 degrees). In another embodiment, the reaction is carried out at a single temperature within the operating parameters of the instrument utilized.

By “quantity threshold method” is meant providing an estimate of quantity based on either exceeding or not exceeding in quantity a comparative.

By “reference” is meant a standard or control condition. As is apparent to one skilled in the art, an appropriate reference is where an element is changed in order to determine the effect of the element.

By “subject” is meant a mammal, including, but not limited to, a human or non-human mammal, such as a bovine, equine, canine, ovine, or feline.

By “target nucleic acid molecule” is meant a polynucleotide to be analyzed. Such polynucleotide may be a sense or antisense strand of the target sequence. The term “target nucleic acid molecule” also refers to amplicons of the original target sequence.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Various aspects of the invention provide vessels, circuits, systems, and method for sample preparation, extraction, and analysis.

### System Overview

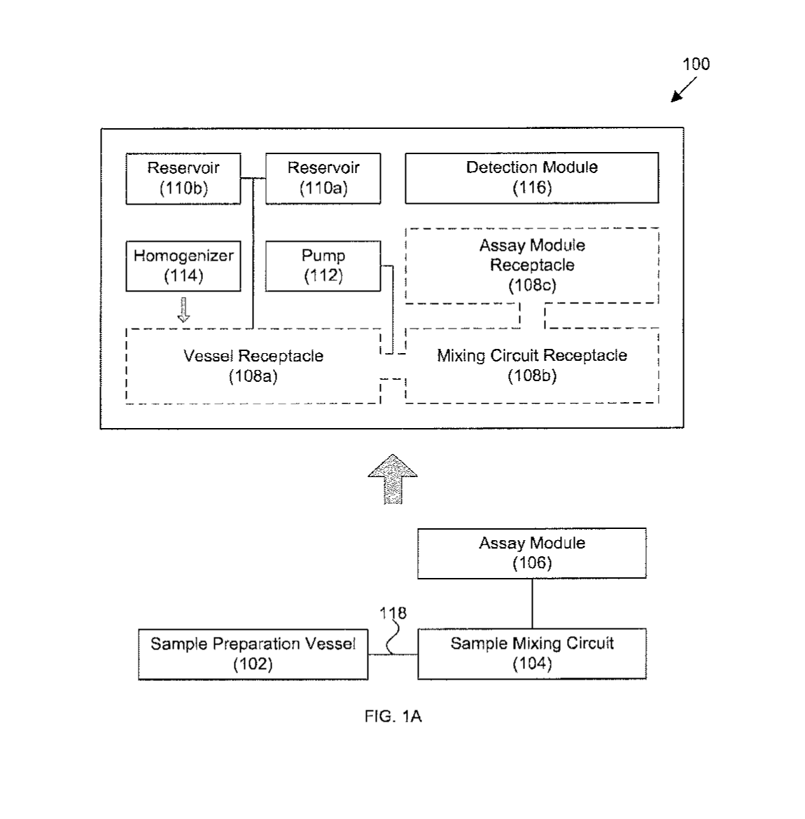

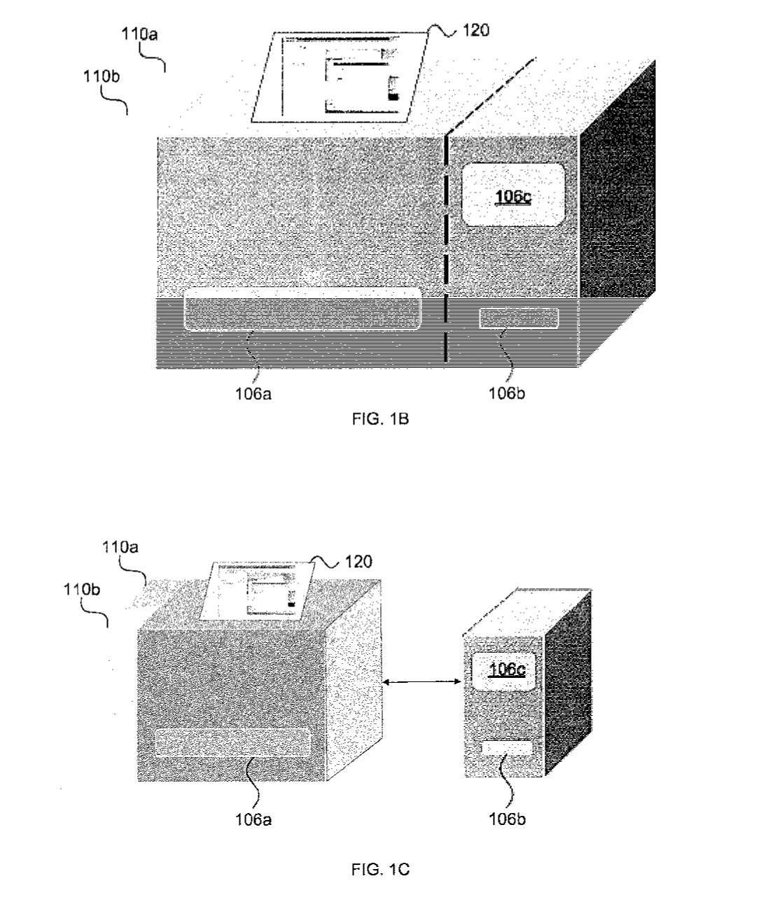

Referring now to FIGS. 1A-1C, one aspect of the invention provides a sample preparation, extraction, and analysis system 100. Embodiments of the system 100 are particularly useful in analyzing samples in the field and can be designed to be particularly rugged and easy to use and clean. For example, the system 100 can include one or more handles for easy transport, a cushioned and/or rubberized case, and/or a plurality of power sources (e.g., batteries).

For example, system 100 can interact with disposable, single-use sample preparation vessels 102, sample mixing circuits 104, and assay modules 106 that confine the processed sample and prevent contamination of other components.

System can further include one or more receptacles 108, fluid reservoirs 110, pumps 112, homogenizers 114, detection modules 116, and/or user interfaces 120 as will be further described herein.

As depicted in FIGS. 1B and 1C, various components of the system 100 can be integrated into a single unit or separate into multiple units that can be physically and/or communicatively coupled.

### Sample Preparation Vessel

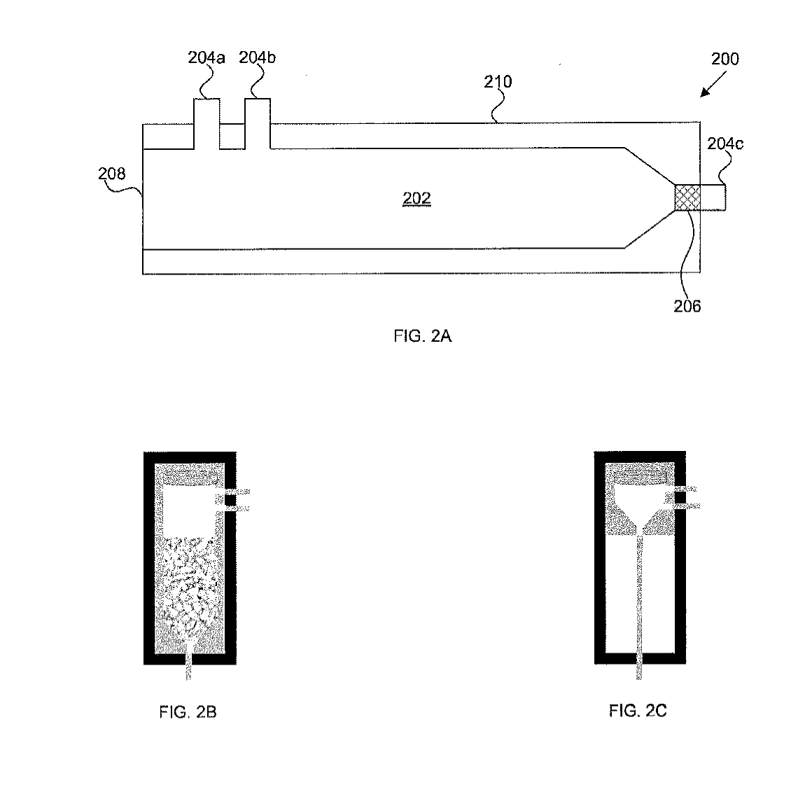

Referring now to FIG. 2A, another embodiment of the invention provides a sample preparation vessel 200. Sample preparation vessel 200 includes a flexible substrate 202, at least one fitting 204, and at least one filter 206 adjacent to the at least one of the fittings 204.

The flexible substrate 202 can be any material capable of substantially retaining a fluid while receiving and translating physical forces from outside the sample preparation vessel 200 to inside the sample preparation vessel 200. Suitable materials include polymers (e.g., flexible polyvinyl chloride), elastomers, and the like. For example, the flexible substrate 202 can be formed from the same or similar material and/or have the same or similar thickness as a plastic storage bag (e.g., about 0.0015 inches, about 0.002 inches, about 0.0025 inches, about 0.003 inches, about 0.004 inches, about 0.005 inches, about 0.006 inches, and the like).

Fittings 204 can include a septum or other sealing device sufficient to hold a vacuum and/or retain a sample before or after processing until the sample preparation vessel 200 is engaged with another component (e.g., the sample mixing circuit 104).

Filter 206 can be any structure capable of preventing particles of an undesirable size from exiting the sample preparation vessel 200 while permitting a fluid to exit the sample preparation vessel 200. A variety of biocompatible filters are available, for example, under the SPECTRA/MESH® trademark from Spectrum Laboratories, Inc. of Rancho Dominguez, Calif. and can be specified by their size selectivity.

The sample preparation vessel 200 can include an opening 208 adapted and configured to receive a sample between the flexible substrate 202. The size of the opening 208 can be configured to accommodate a sample of interest. For example, a relatively small opening 208 (e.g., a substantially elliptical profile of about 3 cm by about 1 cm) could easily receive seeds, while larger openings may be preferred to receive leaves and other larger samples. Opening 208 can be closed through physical, chemical, or thermal means. For example, opening 208 can include a zipper storage mechanism such as those on ZIP-LOC® bags or an adhesive strip. Alternatively, opening 208 can be sealed by thermal, ultrasound, or chemical welding.

In some embodiments, a vacuum sealing device is used to pull a vacuum and then seal the sample preparation vessel 102. Suitable vacuum sealers are available from Accu-Seal Corporation of San Marcos, Calif.

The sample preparation vessel 200 can be formed by bonding two layers of flexible substrate 202 together (e.g., by heat, ultrasound, chemical, other means of welding). In some embodiments, the flexible substrate 202 is bonded to a sidewall member 210 adapted and configured to give thickness and increased volume to the sample preparation vessel 200. Sidewall member 210 can be formed from a variety of rigid or flexible materials such as glass, polymers, plastics, rubbers, and the like.

Sample preparation vessel 200 is advantageously compatible with and agnostic to a variety of samples and quantity of samples. In some embodiments, the internal volume of the sample preparation vessel 200 is varied while maintaining the same footprint as depicted in FIGS. 2B and 2C.

### Sample Mixing Circuits

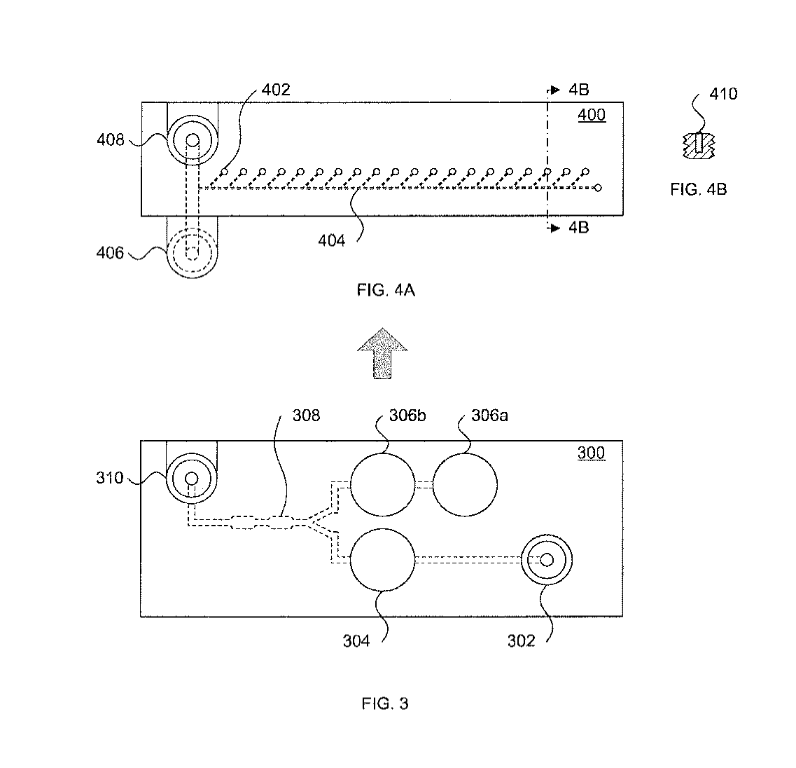

Referring now to FIG. 3, an exemplary sample mixing circuit 300 is depicted. The sample mixing circuit 300 includes a sample ingress port 302, a volumetric sample staging well 304, one or more reagent storage wells 306a, 306b, a sample mixing zone 308, and a sample egress port 310.

Sample ingress port 302 can be any fluidic interface capable of forming a substantially fluid tight seal with a sample source (e.g., outlet fitting 204c of sample preparation vessel 200 or an intermediary). In some embodiments, the sample ingress port 302 can include one or more elastomeric members such as an O-ring or a gasket.

Volumetric sample staging well 304 can be sized to contain an appropriate volume (e.g., a sub-millimeter volume) of the sample relative to the volume(s) of reagents stored in reagent storage wells 306. Such volumes can be specified by the developer of a particular assay.

Reagent storage wells 306 can be pre-loaded with an appropriate volume (e.g., sub-millimeter volumes) of one or more reagents (e.g., PCR Master Mix) common for a plurality of assays in accordance with developer instructions. In one embodiment, reagent storage wells 306 are covered with a blister or other deformable seal as described in U.S. Patent Application Publication No. 2007/0263049. Such a blister or other deformable seal can be compressed, depressed, or otherwise deformed to press fluid out of reagent storage wells 306. In some embodiments, the depression of the blister causes seal under the stored reagent to rupture as described in U.S. Patent Application Publication No. 2007/0263049. In other embodiments, fluid is released from the reagent storage wells 306 by application of air pressure (e.g., from an external source). Use of either architecture allows for sequential release of reagents in order to facilitate various assays.

Sample mixing zone 308 can utilized various geometries and/or mixing elements to facilitate mixing of the sample with one or more reagents. For example, mixing zone 308 can include one or more posts, ridges, baffles, curves, blades, mixers, frits, pellets, and the like.

Sample egress port 310 can be any fluidic interface capable of forming a substantially fluid tight seal with a sink. In some embodiments, the sample ingress port 302 can include one or more elastomeric members such as an O-ring or a gasket.

### Assay Modules

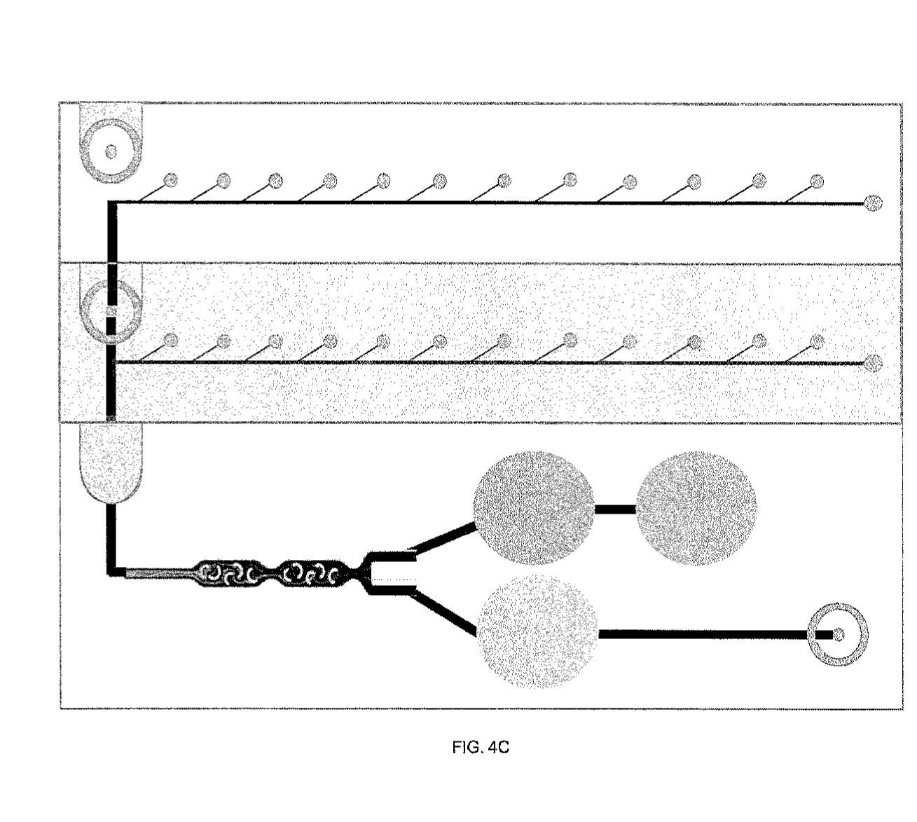

Referring now to FIGS. 4A and 4B, another aspect of the invention provides an assay module 400 including a plurality (e.g., 12) of windows 402 along a fluidic path 404. Assay module 400 can further include an ingress port 406 and an egress port 408.

As seen in FIG. 4B, each window 402 can include one or more oligonucleotides, antibodies, probes, or the like. Such particles can be immobilized (e.g., through covalent bonding) to a substrate and can interact with a sample introduced to the assay module 400 through ingress port 406 within the window 402.

Each window 402 can also include one or more optical interrogation region 410 adapted and configured to facilitate efficient introduction and/or egress of energy (e.g., optical energy) to the window 402. For example, energy of a particular excitation wavelength can be introduced to a window 402 and energy of emitted by a fluorescent probe can be emitted through the interrogation region 410 for detection and analysis.

Assay module 400 can be formed from a variety of materials such as glass, polymers and the like. In some embodiments, the assay module 400 is formed wholly or partially from a material that is opaque (e.g., white or black) or coated with an opaque coating. For example, the assay module 400 can be of a two piece construction in which a bottom piece is opaque and a top piece is transparent.

As depicted in FIGS. 3 and 4A, the assay module 400 can include a complimentary geometry for coupling with the sample mixing circuit 300 and can also be daisy-chained in series to additional assay modules 400. For example, a sample mixing circuit 300 can be coupled to one or more assay modules 400 and fluidically coupled to the sample preparation vessel 200, then placed within receptacles 108 of system 100. Such an assembly is depicted in FIG. 4C.

### Detection Module

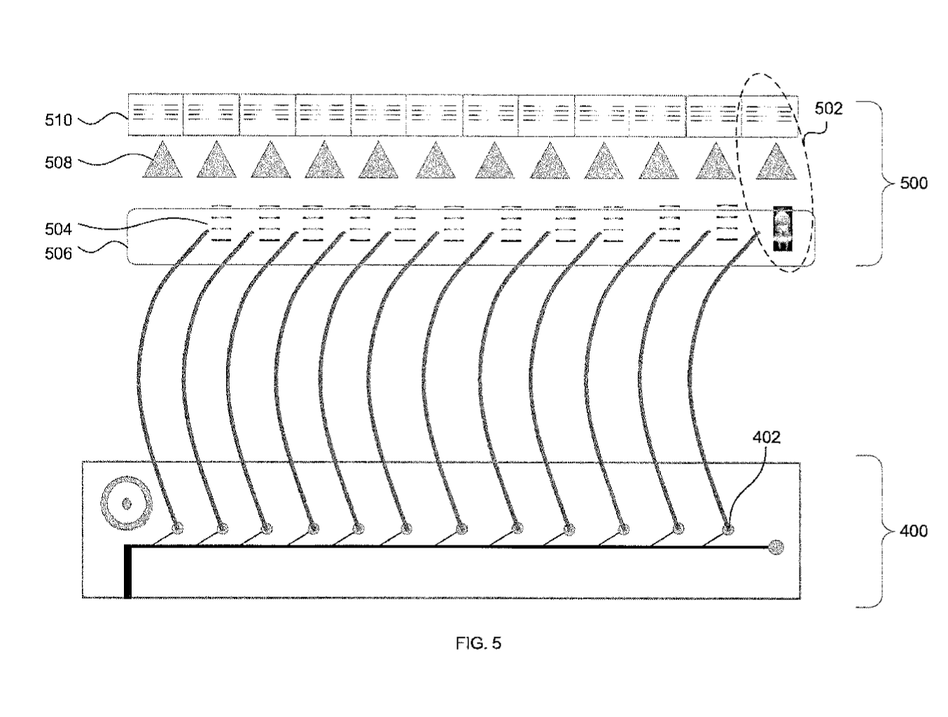

Referring now to FIG. 5, a detection module 500 for interrogating assay module 400 is depicted. The detection module 500 can include an array of detection elements 502, each of which can include one or more optical components such as light sources such as light-emitting diodes (LEDs) 504, beam splitters 506, prisms 508, and optical detectors 510 such as charge-coupled devices (CCDs). Although the use of CCDs provides the most flexibility in supporting various assays, a single color band pass filter corresponding to particular wavelength of interest for a given assay could be used in conjunction with an optical detector 510. The detection elements 502 can be spaced and focused as to interrogate a single window 402 and can be optically shielded in order to eliminate or minimize noise from adjacent windows 402.

### Fabrication

The sample mixing circuit 300 and assay module 400 can be fabricated through a variety of techniques including photolithography negative molding.

### Modularity

Multiple assay modules 400 and detection modules 500 can be utilized in parallel in order to conduct additional assays using a single sample and in a single step. For example, system 100 can initially be sold with a detection module 500, but with one or more expansion interfaces to power and communicate additional detection modules 500 that can be sold separately. For example, 4-8 detection modules 500 could be utilized to conduct 96 assays in parallel.

Likewise, the sample preparation vessel 102, sample mixing circuit 104, and assay module 106 can be used together or separated physically or temporally.

### Sample Homogenization

Referring again to FIG. 1A, sample extraction and analysis system 100 can further include a homogenizer 114 adapted and configured to press against the sample preparation vessel 102 and substantially homogenize the contents thereof. For example, the homogenizer 114 can be adapted and configured to crush the seed coat of a seed or the extracellular matrix of a leaf and expose the inner cells to various processing liquids.

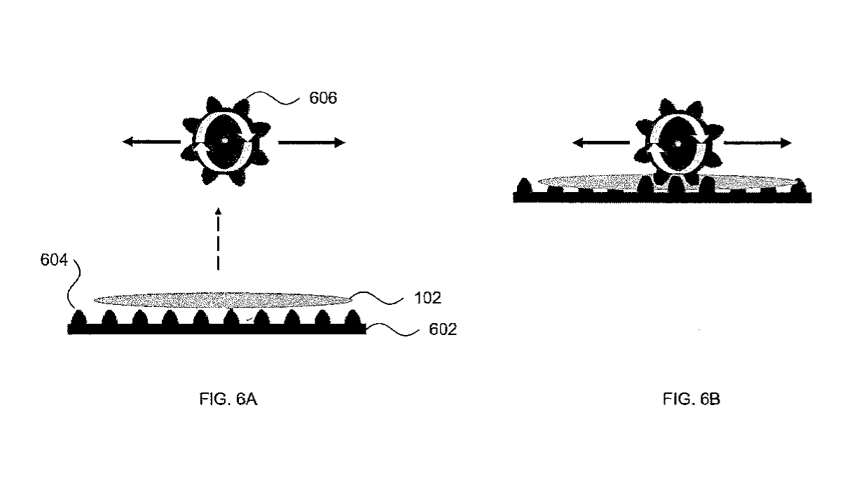

In one embodiment of the invention, a rack-and-pinion gear is used to homogenize the sample as depicted in FIGS. 6A and 6B. For example, the sample preparation vessel 102 can be placed on a platen 602 having a plurality of ridges, studs, or other protrusions or indentations 604. The platen 602 can then be raised toward a pinion 606 having a complimentary profile. The pinion 606 can then rotate in one or both directions to homogenize the sample within the sample preparation vessel 102. In some embodiments, one or more of the platen 602 and the pinion 606 can be heated (e.g., to about 98° C.), cooled, or held at room temperature.

In other embodiments, the homogenizer 106 is a ball-bearing homogenizer such as those available from Bioreba AG of Reinach, Switzerland.

### Sample Preparation

Referring still to FIG. 1, sample extraction and analysis system 100 can further include one or more fluid reservoirs 110 adapted and configured to hold one or more fluids for processing the sample. Each of the fluid reservoirs 108 can be fluidically coupled to an individual ingress port 204 of the sample preparation vessel 102 or can be fluidically coupled to a common ingress port 204 (e.g., via a switching apparatus and/or fluidic multiplexer).

The fluid reservoirs 110 can be maintained at different temperatures. For example, the sample can first be exposed to a fluid having an elevated temperature (e.g., about 95° C.) to macerate the sample, then exposed to a room temperature or cooled fluid. The fluid can be heated using a variety of heaters including resistive (Ohmic or Joule) heating elements. The fluid can be cooled using a variety of elements, such as Peltier thermoelectric cooler. In some embodiments, the same fluid is stored in the fluid reservoirs 110, but maintained at different temperatures.

### Fluid Transfer

Referring still to FIG. 1, a pump 112 can be utilized to transfer the sample-containing fluid from the sample preparation vessel 102 from the sample mixing circuit 104. In one embodiment, the pump 112 is a peristaltic pump, which advantageously permits the use of disposable tubing 118, which could be integral to sample preparation vessel 102.

### Nucleic Acid Amplification Methods

Nucleic acid amplification technologies have provided a means of understanding complex biological processes, detection, identification, and quantification of biological organisms.

The polymerase chain reaction (PCR) is a common thermal cycling dependent nucleic acid amplification technology used to amplify DNA consisting of cycles of repeated heating and cooling of the reaction for DNA melting and enzymatic replication of the DNA using a DNA polymerase. Real-Time quantitative PCR (qPCR) is a technique used to quantify the number of copies of a given nucleic acid sequence in a biological sample. Currently, qPCR utilizes the detection of reaction products in real-time throughout the reaction and compares the amplification profile to the amplification of controls which contain a known quantity of nucleic acids at the beginning of each reaction (or a known relative ratio of nucleic acids to the unknown tested nucleic acid). The results of the controls are used to construct standard curves, typically based on the logarithmic portion of the standard reaction amplification curves. These values are used to interpolate the quantity of the unknowns based on where their amplification curves compared to the standard control quantities.

In addition to PCR, non-thermal cycling dependent amplification systems or isothermal nucleic acid amplification technologies exist including, without limitation: Nicking Amplification Reaction, Rolling Circle Amplification (RCA), Helicase-Dependent Amplification (HDA), Loop-Mediated Amplification (LAMP), Strand Displacement Amplification (SDA), Transcription-Mediated Amplification (TMA), Self-Sustained Sequence Replication (3SR), Nucleic Acid Sequence Based Amplification (NASBA), Single Primer Isothermal Amplification (SPIA), Q-Replicase System, and Recombinase Polymerase Amplification (RPA).

Isothermal nicking amplification reactions have similarities to PCR thermocycling. Like PCR, nicking amplification reactions employ oligonucleotide sequences which are complementary to a target sequences referred to as primers. In addition, nicking amplification reactions of target sequences results in a logarithmic increase in the target sequence, just as it does in standard PCR. Unlike standard PCR, the nicking amplification reactions progress isothermally. In standard PCR, the temperature is increased to allow the two strands of DNA to separate. In nicking amplification reactions, the target nucleic acid sequence is nicked at specific nicking sites present in a test sample. The polymerase infiltrates the nick site and begins complementary strand synthesis of the nicked target nucleotide sequence (the added exogenous DNA) along with displacement of the existing complimentary DNA strand. The strand displacement replication process obviates the need for increased temperature. At this point, primer molecules anneal to the displaced complementary sequence from the added exogenous DNA. The polymerase now extends from the 3′ end of the template, creating a complementary strand to the previously displaced strand. The second oligonucleotide primer then anneals to the newly synthesized complementary strand and extends making a duplex of DNA which includes the nicking enzyme recognition sequence. This strand is then liable to be nicked with subsequent strand displacement extension by the polymerase, which leads to the production of a duplex of DNA which has nick sites on either side of the original target DNA. Once this is synthesized, the molecule continues to be amplified exponentially through replication of the displaced strands with new template molecules. In addition, amplification also proceeds linearly from each product molecule through the repeated action of the nick translation synthesis at the template introduced nick sites. The result is a very rapid increase in target signal amplification; much more rapid than PCR thermocycling, with amplification results in less than ten minutes.

### Nicking Amplification Assays

The invention provides for the detection of target nucleic acid molecules amplified in an isothermal nicking amplification assay. Such assays are known in the art and described herein. See, for example, U.S. Patent Application Publication No. 2009/0081670, International Publication No. 2009/012246, and U.S. Pat. Nos. 7,112,423 and 7,282,328, each of which is incorporated herein in its entirety. Polymerases useful in the methods described herein are capable of catalyzing the incorporation of nucleotides to extend a 3′ hydroxyl terminus of an oligonucleotide (e.g., a primer) bound to a target nucleic acid molecule. Such polymerases include those that are thermophilic and/or those capable of strand displacement. In one embodiment, a polymerase lacks or has reduced 5′-3′ exonuclease activity and/or strand displacement activity. DNA polymerases useful in methods involving primers having 2′-modified nucleotides at the 3′ end include derivatives and variants of the DNA polymerase I isolated from Bacillus stearothermophilus, also taxonomically re-classified as Geobacillus stearothermophilus, and closely related thermophilic bacteria, which lack a 5′-3′ exonuclease activity and have strand-displacement activity. Exemplary polymerases include, but are not limited to the fragments of Bst DNA polymerase I and Gst DNA polymerase I. A nicking enzyme binds double-stranded DNA and cleaves one strand of a double-stranded duplex. In the methods of the invention, the nicking enzyme cleaves the top stand (the strand comprising the 5′-3′ sequence of the nicking agent recognition site). In a particular embodiment of the invention disclosed herein, the nicking enzyme cleaves the top strand only and 3′ downstream of the recognition site. In exemplary embodiments, the reaction comprises the use of a nicking enzyme that cleaves or nicks downstream of the binding site such that the product sequence does not contain the nicking site. Using an enzyme that cleaves downstream of the binding site allows the polymerase to more easily extend without having to displace the nicking enzyme. Ideally, the nicking enzyme is functional under the same reaction conditions as the polymerase. Exemplary nicking enzymes include, but are not limited to, N.Bst9I, N.BstSEI, Nb.BbvCI(NEB), Nb.Bpu10I(Fermantas), Nb.BsmI(NEB), Nb.BsrDI(NEB), Nb.BtsI(NEB), Nt.AlwI(NEB), Nt.BbvCI(NEB), Nt.Bpu10I(Fermentas), Nt.BsmAI, Nt.BspD6I, Nt.BspQI(NEB), Nt.BstNBI(NEB), and Nt.CviPII(NEB). Sequences of nicking enzyme recognition sites are provided at Table 1.

TABLE 1
Nicking enzyme recognition sequences

| | |
|---|---|
| N.Bst9I | 5′-GAGTCNNNNN NN-3′ |
| | \|\|\|\|\|\|\|\|\|\| \|\| |
| | 3′-CTCAGNNNNN•NN-5′ |
| N.BstSEI | 5′-GAGTCNNNNN NN-3′ |
| | \|\|\|\|\|\|\|\|\|\| \|\| |
| | 3′-CTCAGNNNNN•NN-5′ |
| Nb.BbvCI(NEB) | 5′-CCTCA•GC-3′ |
| | \|\|\|\|\| \|\| |
| | 3′-GGAGT CG-5′ |
| Nb.Bpu10I(Fermantas) | 5′-CCTNA•GC-3′ |
| | \|\|\|\|\| \|\| |
| | 3′-GGANT CG-5′ |
| Nb.BsmI(NEB) | 5′-GAATG•CN-3′ |
| | \|\|\|\|\| \|\| |
| | 3′-CTTAC GN-5′ |
| Nb.BsrDI(NEB) | 5′-GCAATG•NN-3′ |
| | \|\|\|\|\|\| \|\| |
| | 3′-CGTTAC NN-5′ |
| Nb.BtsI(NEB) | 5′-GCAGTG•NN-3′ |
| | \|\|\|\|\|\| \|\| |
| | 3′-CGTCAC NN-5′ |
| Nt.AlwI(NEB) | 5′-GGATCNNNN N-3′ |
| | \|\|\|\|\|\|\|\|\| \| |
| | 3′-CCTAGNNNN•N-5′ |
| Nt.BbvCI(NEB) | 5′-CC TCAGC-3′ |
| | \|\| \|\|\|\|\| |
| | 3′-GG•AGTCG-5′ |
| Nt.Bpu10I(Fermentas) | 5′-CC TNAGC-3′ |
| | \|\| \|\|\|\|\| |
| | 3′-GG•ANTCG-5′ |
| Nt.BsmAI | 5′-GTCTCN N-3′ |
| | \|\|\|\|\|\| \| |
| | 3′-CAGAGN•N-5′ |
| Nt.BspD6I | 5′-GAGTCNNNN N-3′ |
| | \|\|\|\|\|\|\|\|\| \| |
| | 3′-CTCAGNNNN•N-5′ |
| Nt.BspQI(NEB) | 5′-GCTCTTCN-3′ |
| | \|\|\|\|\|\|\|\| |
| | 3′-CGAGAAGN-5′ |
| Nt.BstNBI(NEB) | 5′-GAGTCNNNN N-3′ |
| | \|\|\|\|\|\|\|\|\| \| |
| | 3′-CTCAGNNNN•N-5′ |
| Nt.CviPII(NEB) | 5′-CCD-3′ |
| | \|\|\| |
| | 3′-GGH-5′ |

Nicking enzymes also include engineered nicking enzymes created by modifying the cleavage activity of restriction endonucleases (NEB expressions July 2006, vol 1.2), when restriction endonucleases bind to their recognition sequences in DNA, two catalytic sites within each enzyme for hydrolyzing each strand drive two independent hydrolytic reactions which proceed in parallel. Altered restriction enzymes can be engineered that hydrolyze only one strand of the duplex, to produce DNA molecules that are “nicked” (3′-hydroxyl, 5′-phosphate), rather than cleaved. Nicking enzymes may also include modified CRISPR/Cas proteins, Transcription activator-like effector nucleases (TALENs), and Zinc-finger nucleases having nickase activity.

A nicking amplification reaction typically comprises nucleotides, such as, for example, dideoxyribonucleoside triphosphates (dNTPs). The reaction may also be carried out in the presence of dNTPs that comprise a detectable moiety including but not limited to a radiolabel (e.g., ³²P, ³³P, ¹²⁵I, ³⁵S) an enzyme (e.g., alkaline phosphatase), a fluorescent label (e.g., fluorescein isothiocyanate (FITC)), biotin, avidin, digoxigenin, antigens, haptens, or fluorochromes. The reaction further comprises certain salts and buffers that provide for the activity of the nicking enzyme and polymerase.

Advantageously, the nicking amplification reaction is carried out under substantially isothermal conditions where the temperature of the reaction is more or less constant during the course of the amplification reaction. Because the temperature does not need to be cycled between an upper temperature and a lower temperature, the nicking amplification reaction can be carried out under conditions where it would be difficult to carry out conventional PCR. Typically, the reaction is carried out at about between 35 C and 90 C (e.g., about 35, 37, 42, 55, 60, 65, 70, 75, 80, or 85° C.). Advantageously, it is not essential that the temperature be maintained with a great degree of precision. Some variability in temperature is acceptable.

Sets of primers for amplification reactions are selected having G's←15, −16, 17, −18, −19, −20, −25, −30 kcal/mole or more. The performance characteristics of amplification reactions may be altered by increasing the concentration of one or more oligonucleotides (e.g., one or more primers and/or probes) and/or their ratios. High concentrations of primers also favor primer-dimer formation. In various embodiments, concentration of a primers is 100, 200, 300, 400, 500, 600, 700, 800, 900, 1000 nM or more. Melt temperature (Tm) and reaction rate modifiers may also be used to lower the melting temperature of the oligonucleotides, such as (but not limited to) ethylene glycol and glycerol. In addition, DNA polymerase reaction rate modifiers (such as dNTP and magnesium concentration) may be used to alter the reaction rate to lead to a greater quantification precision. In particular embodiments, the 5′ tail sequences of the forward and reverse primers have the same nucleic acid sequence.

This invention provides methods of monitoring a nicking amplification reaction in real time, for example utilizing the amplification strategy as described above. In one embodiment, quantitative nucleic acid amplification utilizes target nucleic acids amplification alongside a control amplification of known quantity. The amount of target nucleic acid can be calculated as an absolute quantification or a relative quantification (semi-quantitative) based on the source of the control (exogenous or endogenous control).

Quantification of the unknown nucleotide sequence can be achieved either through comparison of logarithmic threshold amplification of the unknown to a series of known target sequences in either a separate set of reactions or in the same reaction; or as an internal endogenous or exogenous co-amplification product which produces a threshold value, indicative of either a positive result (if the unknown exceeds the threshold) or negative result (if the unknown does not exceed the threshold).

The invention also provides a method of designing a nicking agent-dependent isothermal strand-displacement amplification assay without experimental screening of a multitude of combinations of candidate forward primers and/or candidate reverse primers. A 35 to 70 bp long region within the target sequence is identified having a 12 to 20 bp sequence in the central portion with a Tm≥the assay temperature (e.g., ˜55° C.). Adjacent sequences 12 bp to 20 bp long immediately downstream and upstream of the 15 to 20 bp long central region are identified, according to the above criteria. The Tm of the chosen double stranded downstream and upstream adjacent sequences deviate from each other by less than ±3° C. A target-specific pair of forward and reverse primers are created by attaching a 5′-tail region for a stable dimer-forming primer to the 5′-terminus of the 12-20 base upstream adjacent sequence and to the 5′-terminus of the complementary strand of the 12-20 base downstream adjacent sequence. When combining the forward primer, reverse primer, and a probe, the primer driving the synthesis of the strand complementary to the probe is in excess over the other primer at a molar ratio of about 1.1:1 to 10:1. The combined concentration of a primer in the assay is no higher than 1000 nM. The assay design method can also be used to convert a pre-validated PCR assay for an amplicon ≤70 bp to an nicking agent-dependent isothermal strand-displacement amplification assay.

### Primer Design

Conventional methods for primer design have focused on primer melting temperature, primer annealing temperature, GC (guaninine and cytosine) content, primer length, and minimizing interactions of the primer with all but the target nucleic acid (see e.g., www.premierbiosoft.com/tech_notes/PCR_Primer_Design.html). Contrary to these methods, it has been found that primers that form stable primer/dimers, expressed in terms of free energy of formation (G), function predictably in nucleic acid amplification reactions. While Free Energy (ΔG) and Melting Temperature (Tm) share primary components Enthalpy (ΔH) and Entropy (ΔS), ΔG and Tm values are derived differently and have no correlative relationship, and the only way to relate a given ΔG with a given Tm value is to explicitly know the value of ΔH and ΔS from which they are derived (Manthey, “mFold, Delta G, and Melting Temperature” ©2005 and 2011 Integrated DNA Technologies). FIGS. 1-11 relate to the design of optimal primers.

The free energy of formation (G) for intermolecular primer structures may be calculated using formulas known in the art. A number of programs are available for determining the formation of various intramolecular and intermolecular primer structures and calculating their G's, including for example mfold and UNAfold prediction algorithms (see e.g., Markham and Zuker. UNAFold: Software for Nucleic Acid Folding and Hybridization. Bioinformatics: Volume 2, Chapter 1, pp 3-31, Humana Press Inc., 2008; Zuker et al. Algorithms and Thermodynamics for RNA Secondary Structure Prediction: A Practical Guide In RNA Biochemistry and Biotechnology, 11-43, NATO ASI Series, Kluwer Academic Publishers, 1999; M. Zuker. Prediction of RNA Secondary Structure by Energy Minimization. Methods in Molecular Biology, 267-294, 1994; Jaeger et al. Predicting Optimal and Suboptimal Secondary Structure for RNA. In Molecular Evolution: Computer Analysis of Protein and Nucleic Acid Sequences, Methods in Enzymology 183, 281-306, 1990; Zuker. On Finding All Suboptimal Foldings of an RNA Molecule. Science 244, 48-52, 1989). OligoAnalyzer 3.1 is one such implementation of mfold for primer design (www.idtdna.com/analyzer/Applications/OligoAnalyzer/). For example with reference to OligoAnalyzer 3.1, G calculations may be performed using the following parameters: Target Type: DNA; Oligo Concentration 0.25 μM; Na⁺ Concentration: 60 mM; Mg⁺⁺ Concentration: 15 mM; and dNTPs Concentration: 0.3 mM.

### 3′ Recognition Region

The invention provides a primer having a 3′ recognition sequence whose primer-target formation is stable (G≤about −20 kcal/mol or more) and has the potential to enhance nucleic acid amplification reaction performance. The 3′ recognition region specifically binds to the a nucleic acid molecule, for example a complementary sequence of the nucleic acid molecule. In certain embodiments, the 3′ recognition region has a sequence that is complementary to 12, 13, 14, 15, 16, 17, 18, 19, or 20 bases or more of a nucleic acid sequence. In particular embodiments, the 3′ recognition region comprises one or more inosine bases. In specific embodiments, the 3′ recognition region comprises no more than 2/12 inosines. In various embodiments, the primer-target melting temperature is equal to or greater than 8° or 6° C. below the reaction or extension temperature of the assay (Tm≥assay temperature −8°). In particular embodiments, the 3′ recognition sequence comprises 12-20, 12-17, or 12-14 bases. In particular embodiments, the primer-target formation is more stable than self dimer formation (e.g., G≤about −15, −16, −17, −18, −19, −20 kcal/mol or more). Preferably, the 3′ recognition sequence does not contain self-complementary sequences, short inverted repeats (e.g., >4 bases/repeat), or sequences that otherwise promote intramolecular interactions, which have the potential to interfere with primer-target annealing.

In particular, a primer of the invention having a 3′ recognition sequence is useful in nicking amplification assays. Additionally, the target specific 3′ recognition region comprises one or more 2′ modified nucleotides (e.g., 2′-O-methyl, 2′-methoxyethoxy, 2′-fluoro, 2′-alkyl, 2′-allyl, 2′-O-[2-(methylamino)-2-oxoethyl], 2′-hydroxyl (RNA), 4′-thio, 4′-CH₂—O-2′-bridge, 4′-(CH₂)₂—O-2′-bridge, and 2′-O-(N-methylcarbamate)). Without being bound to theory, it is hypothesized that incorporating one or more 2′ modified nucleotides in the recognition regions reduces or eliminates intermolecular and/or intramolecular interactions of primers/templates (e.g., primer-dimer formation), and, thereby, reduces or eliminates the background signal in isothermal amplification. The 2′ modified nucleotide preferably has a base that base pairs with the target sequence. In particular embodiments, two or more 2′ modified nucleotides (e.g., 2, 3, 4, 5 or more 2′ modified nucleotides) in the target specific recognition region are contiguous (e.g., a block of modified nucleotides). In some embodiments, the block of 2′ modified nucleotides is positioned at the 3′ end of the target specific recognition region. In other embodiments, the block of 2′ modified nucleotides is positioned at the 5′ end of the target specific recognition region. When the block of 2′ modified nucleotides is positioned at the 5′ end of the target specific recognition region, the 2′ modified nucleotides may be separated from the nick site by one or more non-modified nucleotides (e.g., 2, 3, 4, 5 or more 2′ unmodified nucleotides). Applicants have found that positioning of one or more 2′ modified nucleotides or of a block of 2′ modified nucleotides alters the kinetics of amplification. When the one or more 2′ modified nucleotides or block of 2′ modified nucleotides are positioned at or near the 5′ end of the recognition region or proximal to the nick site, real-time amplification reactions showed decreased time to detection. Additionally, the signal curve is contracted and the slope of the curve shifted.

In a related embodiment, ratios of a primer having one or more 2′ modified nucleotides can be used to alter the time-to-detection and/or the efficiency of the reaction for the ‘tuning’ of reactions, resulting in a predictable control over reaction kinetics. Increasing the ratio of primer having one or more 2′ modified nucleotides at the 3′ end of the recognition sequence to primer having one or more 2′ modified nucleotides at the 5′ end of the recognition sequence contracted the signal curve and shifted the slope of the curve. It is advantageous to be able to “tune” a reaction providing a means to manipulate both the time-to-detection as well as the efficiency of the reaction. Relative quantification using an internal control requires that two important conditions be met. First, it is beneficial to be able to modify a reaction's time-to-detection creating a non-competitive reaction condition. Thus, by affecting the control reaction to be detectable at a later time-point (relative to the target of interest) the control reaction does not out-compete the specific target of interest even when the target of interest is in low initial abundance. Second, to ensure a true relative abundance calculation, it is required that the control and specific target reactions have matched efficiencies. By controlling the efficiency of each reaction using a “tuning” condition enables reactions to be matched allowing for satisfactory relative quantification calculations. Tuning the reactions can be used to match efficiencies of target nucleic acid amplification and reference nucleic amplification (e.g., internal standard) in quantitative PCR (qPCR). Additionally, amplification curves of the target nucleic acid and the internal standard may be altered so time of detection of their amplification products are separated, while providing the same efficiency for target nucleic acid amplification and internal standard amplification. Through the use of specific combinations and ratios of oligonucleotide structures within a reaction it is possible to create conditions which enable tuned reaction performance.

### 5′ Tail Dimerization Region

The invention provides a primer having a 5′ tail region capable of self-dimerization that enhances nucleic acid amplification reaction performance. Without being bound to theory, in a nucleic acid amplification reaction the primer anneals to the target nucleic acid as a primer-dimer. For example, nicking amplification primers have a nicking agent recognition site present at the 5′ end that is unrelated to the binding specificity of the primer for the target recognition sequence. Non-specific background products from non-specific primer interactions have the potential to sequester reaction components that would otherwise have been utilized for the amplification of the specific product. In various embodiments, homodimer formation is stable (e.g., G≤about −30, −35, −40, −45, −50, −55, −60 kcal/mol or more). In various embodiments, the homodimer has a melting temperature higher than the extension reaction temperature. In particular embodiments, the 5′ tail region has a sequence that is a palindrome. In further embodiments, the 5′ tail region is at least 12 bases (e.g., 12, 13, 14, 15, 16, 17, 18, 19, 20, 21, 22, 23, 24 bases) in length. In additional embodiments, the 5′ tail region has a GC content of 80-90%. In certain embodiments, homodimer formation is more stable than formation of other less stable primer dimer conformations formation (e.g., G≤about −12, −13, −14, −15, −16, −17, −18, −19, −20, −25, −30, −35, −40 kcal/mol or more).

In particular, a primer of the invention having a 5′ tail sequence is useful in nicking amplification reactions. For use in nicking amplification reactions, the 5′ tail region comprises one or more nicking agent recognition sites and the 5′ tail region has a symmetrically inverted sequence. In particular embodiments, the 5′ tail region contains an even number of nucleotides (e.g., 22, 24 nucleotides). The nick site is designed to be positioned between the nucleotide at the 3′ end of the 5′ tail region and the nucleotide at the 5′ end of the 3′ recognition region. Without being bound to theory, the nicking enzyme does not cleave at the nick site when the 3′ recognition is single-stranded. However, cleavage at the nick site occurs when the 3′ recognition region is double stranded (e.g., when the primer is incorporated into a double-stranded target nucleic acid molecule during the course of the nucleic acid amplification reaction).

In various embodiments, the 5′ tail sequence comprises from 5′ to 3′ an inverted nicking enzyme recognition sequence that is operatively linked to a palindromic sequence (or self-complementary sequence) that is operatively linked to a nicking enzyme recognition sequence. In certain embodiments, the spacer region is an even number of nucleotides (e.g., 2, 4, 6, etc.). Exemplary 5′ tails based on the Nt.BstNBI nicking enzyme recognition sequence (5′-GAGTC-3′) having a 2, 4, and 6 nucleotide spacers comprise a nucleic acid sequences according to the formula below

5′-GACTCN₁N_{1′}GAGTC-3′
5′-GACTCN₂N₁N_{1′}N_{2′}GAGTC-3′
5′-GACTCN₃N₂N₁N_{1′}N_{2′}N_{3′}GAGTC-3′

where “N” is any nucleotide (e.g., having an adenine (A), thymine (T), cytosine (C), or guanine (G) nucleobase), and N₁ is complementary to N_{1′}, N₂ is complementary to N_{2′}, and N₃ is complementary to N_{3′}, etc.

Exemplary 5′ tail region sequences 24 nucleotides in length having a Nt.BstNBI recognition sequence can be generated based on the following template 5′-NNNNGACTCNNNNNNGAGTCNNNN-3′. Based on this template, there are 537,824 5′ tail sequences having the following properties: G=−48 Kcal/mole to −62 kcal/mole; G<−40 kcal/mole; and GC content 68% to 84%. Of these, 1050 selected sequences are provided, representing 0.2% of the entire sequence space (248,832). Exemplary 5′ tail region sequences 22 nucleotides in length having a Nt.BstNBI recognition sequence and based on the following template 5′-NNNNGACTCNNNNGAGTCNNNN-3′. Based on this template, there are 248,832 5′ tail sequences having the following properties: G=−47 Kcal/mole to −55 kcal/mole; G<−40 kcal/mole; and GC content 72% to 82%. Of these, 200 selected sequences are provided, representing 0.08% of the entire sequence space (248,832).

### Target Nucleic Acid Molecules

Methods and compositions of the invention are useful for the identification of a target nucleic acid molecule in a test sample. The target sequences is amplified from virtually any samples that comprises a target nucleic acid molecule, including but not limited to samples comprising fungi, spores, viruses, or cells (e.g., prokaryotes, eukaryotes). Exemplary test samples include environmental samples, agricultural products (e.g., seeds) or other foodstuffs and their extracts, and DNA identification tags. Exemplary test samples include biological samples, body fluids (e.g. blood, serum, plasma, amniotic fluid, sputum, urine, cerebrospinal fluid, lymph, tear fluid, feces, or gastric fluid), tissue extracts, organs, culture media (e.g., a liquid in which a cell, such as a pathogen cell, has been grown). If desired, the sample is purified prior to inclusion in a NEAR reaction using any method typically used for isolating a nucleic acid molecule from a biological sample.

In one embodiment, primer/template oligonucleotides amplify a target nucleic acid of a pathogen to detect the presence of a pathogen in a sample. Exemplary pathogens include fungi, bacteria, viruses and yeast. Such pathogens may be detected by identifying a nucleic acid molecule encoding a pathogen protein, such as a toxin, in a test sample. Exemplary toxins include, but are not limited to aflatoxin, cholera toxin, diphtheria toxin, Salmonella toxin, Shiga toxin, Clostridium botulinum toxin, endotoxin, and mycotoxin. For environmental applications, test samples may include water, liquid extracts of air filters, soil samples, building materials (e.g., drywall, ceiling tiles, wall board, fabrics, wall paper, and floor coverings), environmental swabs, or any other sample.

In one embodiment disclosed herein, primer/template oligonucleotides amplify a target nucleic acid of a plant (e.g., used as an internal control in molecular breeding experiments geared towards improving, for example, the plant's resistance to drought, the plant's resistance to herbicides, and/or to predation by harmful insects). Seeds (e.g., soybeans) are an exemplary plant test sample.

Target nucleic acid molecules include double-stranded and single-stranded nucleic acid molecules (e.g., DNA, RNA, and other nucleobase polymers known in the art capable of hybridizing with a nucleic acid molecule described herein). RNA molecules suitable for detection with a detectable oligonucleotide probe or detectable primer/template oligonucleotide of the invention include, but are not limited to, double-stranded and single-stranded RNA molecules that comprise a target sequence (e.g., messenger RNA, viral RNA, ribosomal RNA, transfer RNA, microRNA and microRNA precursors, and siRNAs or other RNAs described herein or known in the art). DNA molecules suitable for detection with a detectable oligonucleotide probe or primer/template oligonucleotide of the invention include, but are not limited to, double stranded DNA (e.g., genomic DNA, plasmid DNA, mitochondrial DNA, viral DNA, and synthetic double stranded DNA). Single-stranded DNA target nucleic acid molecules include, for example, viral DNA, cDNA, and synthetic single-stranded DNA, or other types of DNA known in the art.

In general, a target sequence for detection is between 10 and 100 nucleotides in length (e.g., 10, 15, 20, 25, 30, 35, 40, 45, 50, 60, 70, 80, 90, 100 nucleotides. The GC content of the target nucleic acid molecule is selected to be less than about 45, 50, 55, or 60%. Desirably, the target sequence and nicking enzymes are selected such that the target sequence does not contain nicking sites for any nicking enzymes that will be included in the reaction mix.

### Detectable Oligonucleotide Probes

The present invention provides for the quantitative detection of target nucleic acid molecules or amplicons thereof in a nicking amplification reaction using non-amplifiable detectable polynucleotide probes comprising at least one polymerase-arresting molecule (e.g., nucleotide modification or other moiety that renders the oligonucleotide capable of binding a target nucleic acid molecule, but incapable of supporting template extension utilizing the detectable oligonucleotide probe as a target). Without wishing to be bound by theory, the presence of one or more moieties which does not allow polymerase progression likely causes polymerase arrest in non-nucleic acid backbone additions to the oligonucleotide or through stalling of a replicative polymerase (i.e. C3-spacer, damaged DNA bases, other spacer moiety, O-2-Me bases). These constructs thus prevent or reduce illegitimate amplification of the probe during the course of a nicking amplification reaction. This distinguishes them from conventional detection probes, which must be added at the end of the nicking amplification reaction to prevent their amplification.

Conventional detection probes have proven impractical for quantitating a nicking amplification reaction in real time. If conventional detection probes are incorporated into the nicking amplification reaction, these conventional detection probes are amplified concurrently with the target. The amplification of these detection molecules masks the detection of legitimate target amplicons due to the number of starting molecules of the detection probe at the start of the reaction.

The invention provides non-amplifiable detectable polynucleotide probe that comprise least one polymerase-arresting molecule. A polymerase-arresting molecule of the invention includes, but is not limited to, a nucleotide modification or other moiety that blocks template extension by replicative DNA polymerases, thereby preventing the amplification of detection molecules; but can allow proper hybridization or nucleotide spacing to the target molecule or amplified copies of the target molecule. In one embodiment, a detectable oligonucleotide probe of the invention comprises a 3 carbon spacer (C3-spacer) that prevents or reduces the illegitimate amplification of a detection molecule.

In one embodiment, a detectable oligonucleotide probe comprises one or more modified nucleotide bases having enhanced binding affinity to a complementary nucleotide. Examples of modified bases include, but are not limited to 2′ Fluoro amidites, and 2′OMe RNA amidites (also functioning as a polymerase arresting molecule). Detectable oligonucleotide probes of the invention can be synthesized with different colored fluorophores and may be designed to hybridize with virtually any target sequence. In view of their remarkable specificity, a non-amplifiable detectable polynucleotide probe of the invention is used to detect a single target nucleic acid molecule in a sample, or is used in combination with detectable oligonucleotide probes each of which binds a different target nucleic acid molecule. Accordingly, the non-amplifiable detectable polynucleotide probes of the invention may be used to detect one or more target nucleic acid molecules in the same reaction, allowing these targets to be quantitated simultaneously. The present invention encompasses the use of such fluorophores in conjunction with the detectable oligonucleotide probes described herein.

Implementation in Hardware and/or Software

The methods described herein can be implemented on general-purpose or specially programmed hardware or software. For example, the methods can be implemented by a computer readable medium. Accordingly, the present invention also provides a software and/or a computer program product configured to perform the algorithms and/or methods according to any embodiment of the present invention. It is well-known to a skilled person in the art how to configure software which can perform the algorithms and/or methods provided in the present invention. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like). The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. (See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.) Additionally, the present invention may have preferred embodiments that include methods for providing genetic information over networks such as the Internet.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

This application may be related to International Application No. PCT/US2013/035750, filed Apr. 9, 2013, which claims the benefit of U.S. Provisional Patent Application No. 61/621,975, filed Apr. 9, 2012, the entire contents of which are incorporated herein by reference.

This application may be related to International Application No. PCT/US2011/047049, filed Aug. 9, 2011, which claims the benefit of U.S. Provisional Patent Application No. 61/373,695, filed Aug. 13, 2010, the entire contents of which are incorporated herein by reference.

1. A sample preparation vessel comprising:
a flexible substrate defining at least one sealable opening adapted and configured to receive a solid sample;
at least one fitting; and
at least one filter adjacent to the at least one fitting, the filter adapted and configured to permit extracted fluids to exit the vessel while retaining solid particles.

2. A microfluidic circuit comprising:
a fluidic path;
a first row of windows, each window including a chamber and an optical lens dome on a first surface of the microfluidic circuit; and
an outlet adapted and configured for coupling with additional rows of windows.

3. A system comprising:
a first port in fluid communication with at least one fluid reservoir and adapted and configured for removable coupling with a sample preparation vessel, the one or more ports collectively;
a second port adapted and configured to receive a sample from a sample mixing circuit;
a first receptacle adapted and configured to receive the sample preparation vessel; and
a second receptacle adjacent to the first receptacle, the second receptacle adapted and configured to receive the sample mixing circuit and hold the sample mixing circuit in fluid communication with the sample preparation vessel.

4. The system of claim 3, further comprising a homogenizer adapted and configured to press against the sample preparation vessel and substantially homogenize the contents thereof.

5-9. (canceled)

10. A method for extracting an analyte from a sample, the method comprising:
introducing the sample into a sample preparation vessel according to claim 1; and
mixing the sample with a buffer capable of extracting and/or solubilizing the analyte in the sample preparation vessel, thereby extracting an analyte from a sample.

11. (canceled)

12. The method of claim 10, wherein the sample or solid sample is a biological sample or an environmental sample.

13. The method of claim 10, wherein the sample or solid sample is a seed, plant tissue, or plant part.

14-15. (canceled)

16. A method of detecting a target nucleic acid molecule, the method comprising:
introducing a sample comprising a target nucleic acid molecule into the mixing chamber of the microfluidic circuit of claim 2, wherein the mixing chamber comprises one or more reagents for amplifying the target nucleic acid; and
detecting the target nucleic acid molecule in a window of the microfluidic circuit.

17. The method of claim 16, wherein the microfluidic circuit comprises one or more blisters in fluid connection with the mixing chamber, wherein compression of one or more blisters introduce one or more reagents into the mixing chamber.

18. The method of claim 16, wherein the reagents comprise one or more of a nickase, DNA polymerase, RNA polymerase, dNTPs, primer, probe, enzyme, and/or reaction buffer.

19. (canceled)

20. The method of claim 16, wherein the reaction is by PCR, qPCR, an isothermal nucleic acid amplification reaction, Nicking and Extension Amplification Reaction (NEAR), Rolling Circle Amplification (RCA), Helicase-Dependent Amplification (HDA), Loop-Mediated Amplification (LAMP), Strand Displacement Amplification (SDA), Transcription-Mediated Amplification (TMA), Self-Sustained Sequence Replication (3SR), Nucleic Acid Sequence Based Amplification (NASBA), Single Primer Isothermal Amplification (SPIA), Q-β Replicase System, or Recombinase Polymerase Amplification (RPA).

21. A method of detecting an analyte in a sample, the method comprising:
extracting an analyte from a sample in the sample preparation vessel of the system of claim 3;
mixing the analyte and one or more reagents in the sample mixing circuit of the system; and
detecting the analyte using an optical imaging device of the system.

22. A method of detecting one or more analytes in a sample, the method comprising:
extracting the one or more analytes from the sample in the sample preparation vessel of the system of claim 3;
mixing the analytes and one or more preparation reagents in the sample mixing circuit of the system;
introducing the mixture of analytes and preparation reagents into an array of chambers or windows comprising one or more detection reagents; and
detecting the analytes using the array of optical imaging devices of the system.

23. A method of detecting a target nucleic acid molecule in a sample, the method comprising:
extracting the target nucleic acid molecule from a sample in the sample preparation vessel of the system of claim 3;
mixing the target nucleic acid molecule and one or more reagents in the sample mixing circuit of the system;
amplifying the target nucleic acid molecule; and
detecting the analyte using an optical imaging device of the system.

24. A method of detecting one or more target nucleic acid molecules in a sample, the method comprising:
extracting the one or more target nucleic acid molecules from the sample in the sample preparation vessel of the system of claim 3;
mixing the one or more target nucleic acid molecules and one or more preparation reagents in the sample mixing circuit of the system;
introducing the mixture of target nucleic acid molecules and preparation reagents into an array of chambers or windows comprising one or more amplification and/or detection reagents;
amplifying the target nucleic acid molecules in the array of chambers or windows; and
detecting the analytes using the array of optical imaging devices of the system.

25. The method of claim 23, wherein the reagents comprise one or more of a nickase, DNA polymerase, RNA polymerase, dNTPs, primer, probe, enzyme, and/or reaction buffer.

26. The method of claim 23, wherein the target nucleic acid is DNA or RNA.

27. The method of claim 23, wherein the amplifying is by PCR, qPCR, an isothermal nucleic acid amplification reaction, Nicking and Extension Amplification Reaction (NEAR), Rolling Circle Amplification (RCA), Helicase-Dependent Amplification (HDA), Loop-Mediated Amplification (LAMP), Strand Displacement Amplification (SDA), Transcription-Mediated Amplification (TMA), Self-Sustained Sequence Replication (3SR), Nucleic Acid Sequence Based Amplification (NASBA), Single Primer Isothermal Amplification (SPIA), Q-3 Replicase System, or Recombinase Polymerase Amplification (RPA).

28. The method of claim 23, wherein each chamber or window comprises a set of nucleic acid primers for amplifying the target nucleic acid.

29. The method of claim 23, wherein each chamber or window comprises a fluorescently labeled nucleic acid probe for detecting the target nucleic acid.


SEQUENCE LISTING (11 sequences)

SEQ ID NO: 1 - DNA, 12 bp, Artificial Sequence
  Description of Artificial Sequence Synthetic oligonucleotide
  modified_base (6)..(12): a, c, t, g, unknown or other
gagtcnnnnnnn

SEQ ID NO: 2 - DNA, 12 bp, Artificial Sequence
  Description of Artificial Sequence Synthetic oligonucleotide
  modified_base (1)..(7): a, c, t, g, unknown or other
nnnnnnngactc

SEQ ID NO: 3 - DNA, 10 bp, Artificial Sequence
  Description of Artificial Sequence Synthetic oligonucleotide
  modified_base (6)..(10): a, c, t, g, unknown or other
ggatcnnnnn

SEQ ID NO: 4 - DNA, 10 bp, Artificial Sequence
  Description of Artificial Sequence Synthetic oligonucleotide
  modified_base (1)..(5): a, c, t, g, unknown or other
nnnnngatcc

SEQ ID NO: 5 - DNA, 10 bp, Artificial Sequence
  Description of Artificial Sequence Synthetic oligonucleotide
  modified_base (6)..(10): a, c, t, g, unknown or other
gagtcnnnnn

SEQ ID NO: 6 - DNA, 10 bp, Artificial Sequence
  Description of Artificial Sequence Synthetic oligonucleotide
  modified_base (1)..(5): a, c, t, g, unknown or other
nnnnngactc

SEQ ID NO: 7 - DNA, 12 bp, Artificial Sequence
  Description of Artificial Sequence Synthetic oligonucleotide
  modified_base (6)..(7): a, c, t, g, unknown or other
  See specification as filed for detailed description of substitutions and preferred embodiments
gactcnngagtc

SEQ ID NO: 8 - DNA, 14 bp, Artificial Sequence
  Description of Artificial Sequence Synthetic oligonucleotide
  modified_base (6)..(9): a, c, t, g, unknown or other
  See specification as filed for detailed description of substitutions and preferred embodiments
gactcnnnngagtc

SEQ ID NO: 9 - DNA, 16 bp, Artificial Sequence
  Description of Artificial Sequence Synthetic oligonucleotide
  modified_base (6)..(11): a, c, t, g, unknown or other
  See specification as filed for detailed description of substitutions and preferred embodiments
gactcnnnnnngagtc

SEQ ID NO: 10 - DNA, 24 bp, Artificial Sequence
  Description of Artificial Sequence Synthetic oligonucleotide
  modified_base (1)..(4): a, c, t, g, unknown or other
  modified_base (10)..(15): a, c, t, g, unknown or other
  modified_base (21)..(24): a, c, t, g, unknown or other
nnnngactcnnnnnngagtcnnnn

SEQ ID NO: 11 - DNA, 22 bp, Artificial Sequence
  Description of Artificial Sequence Synthetic oligonucleotide
  modified_base (1)..(4): a, c, t, g, unknown or other
  modified_base (10)..(13): a, c, t, g, unknown or other
  modified_base (19)..(22): a, c, t, g, unknown or other
nnnngactcnnnngagtcnnnn